(19) United States
(12) Patent Application Publication
(10) Pub. No.: US 2004/0158250 A1
(43) Pub. Date: Aug. 12, 2004

(54) Anterior cervical corpectomy plate

(76) Inventor: James L. Chappuis, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/662,488

(22) Filed: Sep. 15, 2003

Related U.S. Application Data
(60) Provisional application No. 60/410,783, filed on Sep. 13, 2002.

(51) Int. Cl. A61B 17/56

(52) U.S. Cl. 606/69

(57) ABSTRACT

Anterior cervical corpectomy plates are provided. An exemplary place comprises a fixing member for fixing the plate to a portion of a cervical spine and an adjustable member for adjusting a length of the plate.

### CROSS-REFERENCE TO RELATED APPLICATION

This application is based on and claims priority to co-pending U.S. provisional patent application entitled, “Anterior Cervical Corpectomy Plate,” having serial No. 60/410,783, filed Sep. 13, 2002, which is entirely incorporated herein by reference.

### TECHNICAL FIELD

The present invention generally relates to surgical instruments and, in particular, relates to anterior cervical corpectomy plates.

### DESCRIPTION OF THE RELATED ART

The human spine is composed of a column of thirty-three bones, called vertebra, and their adjoining structures. The twenty-four vertebrae nearest the head are separate bones capable of individual movement and are generally connected by anterior and posterior longitudinal ligaments and by discs of fibrocartilage, called intervetebral discs, positioned between opposing faces of adjacent vertebrae. The twenty-four vertebrae are commonly referenced in three sections. The cervical spine, closest to the head and often referenced as the “neck,” comprises the first seven vertebrae of the spine. The thoracic spine and the lumbar spine are below the cervical spine. Each of the vertebra include a vertebral body and a dorsal arch, which enclose an opening, called the vertebral foramen, through which the spinal cord and the spinal nerve pass. The remaining nine vertebrae below the lumbar spine are fused to form the sacrum and the coccyx and are incapable of individual movement.

Degenerative changes in the cervical spine are not uncommon. Such changes are often caused by the intervertebral discs wearing out and beginning to collapse or herniate, and becoming less flexible. Common causes of cervical spine disorders include arthritis, injuries or trauma, and in some cases spinal cord compression, tumors, or spinal infection. Pain caused by these and other maladies can be lessened or eliminated by a cervical corpectomy.

A cervical corpectomy is the removal of vertebral bodies and surrounding intervertebral discs which are causing pressure on the spinal cord. Upon removal, autograft, or allograft bone or spacer is disposed in the void left by the removed material. Once the graft is disposed in the opening, the cervical spine is stabilized, typically with either a cervical collar (brace) or a metal plate, to help promote fusion of the graft to remaining vertebrae. Typically, a metal plate of non-variable length is screwed into the vertebra above and below the graft, usually with two screws at each contact location. Loosening of an attached plate is not uncommon, especially in long fusion, with such little anchorage of the plate. These plates also either fail to allow for any settling, or they allow for very limited, but not controlled, settling.

Current translation plates that do allow for settling have elongated, or ovular, apertures through which the screws fixing the plate to the vertebrae above and below the bone graft are disposed. As settling occurs, the plate slides along the screws as allowed by the length of the aperture. As such, where settling occurs, the plate used is longer than necessary resulting in excess plate length extending above and/or below the bone graft.

Thus, a heretofore undaddressed need exists in the industry to address the aforementioned deficiencies and inadequacies.

### SUMMARY

Anterior cervical corpectomy plates are provided. An embodiments of the present invention provide an anterior cervical corpectomy plate comprises a fixing member for fixing the plate to a portion of a cervical spine and an adjustable member for adjusting a length of the plate.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

Many aspects can be better understood with reference to the following drawings. The components in the drawings are not necessarily to scale. Moreover, in the drawings, like reference numerals designate corresponding parts throughout the several views.

FIG. 1 illustrates a perspective view of an embodiment of an anterior cervical corpectomy plate.

FIG. 2 illustrates a side view of the embodiment of the anterior cervical corpectomy plate illustrated in FIG. 1 mounted in position.

### DETAILED DESCRIPTION OF THE PREFERRED EMBODIMENTS

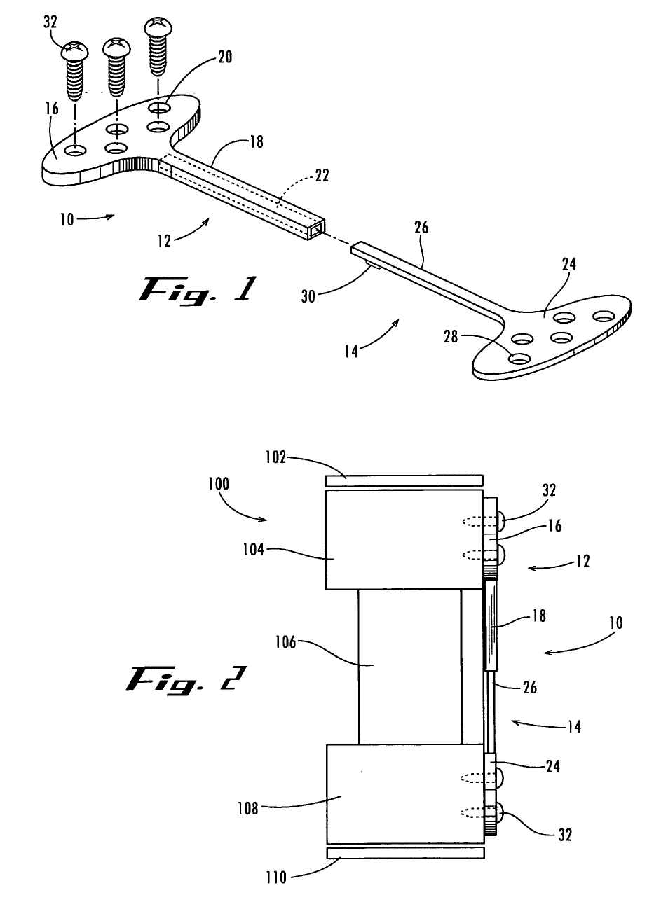

FIG. 1 illustrates one preferred embodiment of an anterior cervical corpectomy plate 10. FIG. 2 illustrates the plate 10 mounted in position on a portion of a cervical spine 100.

More specifically, and with reference to FIG. 1, the anterior cervical corpectomy plate 10 (hereinafter, “the plate”) comprises a first plate member 12 and a second plate member 14. First plate member 12 comprises a first mount 16 and a first support member 18 extending therefrom. The first mount 16 comprises a plurality of apertures 20 disposed therethrough. Although five apertures 20 are illustrated, it should be understood that any number of apertures 20 can be included. However, it is desirable that a plurality of apertures 20 are included for increased anchor strength of the plate 10. The first support member 18 comprises a substantially rigid, elongated channel 22.

Second plate member 14 comprises a second mount 24 and a second support member 26 extending therefrom. The second mount 24 comprises a plurality of apertures 28 disposed therethrough. Although five apertures 28 are illustrated, it should be understood that any number of apertures 28 can be included. However, it is desirable that a plurality of apertures 28 are included for increased anchor strength of the plate 10. The apertures 20 of the first mount 16 and the apertures 28 of the second mount 24 are adapted to each receive a screw 32 therein for fixing the plate 10 in position, as illustrated in FIG. 2.

Second support member 26 is a substantially rigid, elongated member adapted to engage and slide axially within the channel 22 of the first support member 18. It should be understood that the first support member 18 having a channel 22 disposed therein is one example of a configuration providing the desired length variable plate 10. It should be noted that the variable length attribute of the plate 10 can be achieved through various configurations, all of which are within the spirit of the present invention.

A stop 30 can be included on the plate 10. As illustrated herein, the stop 30 is disposed on the second support member 26. It should be understood that the stop 30 can be disposed in any suitable location on the plate 10. The stop 30 limits the length to which the plate 10 can be decreased thereby limiting the amount of settling of an inserted graft 106 (FIG. 2). Upon the plate 10 reaching the minimum desired length, the stop 30 engages the end of the channel 22 thereby preventing further axial movement of the first support member 18 and the second support member 26 with respect to each other. It is desirable that the stop 30 can be disposed at varying locations along the second support member 26 thereby allowing a surgeon to select the appropriate amount of settling for the patient. The stop 30 can also be stationary.

In one configuration, the stop 30 can comprise a pin and row of apertures along the length of the second support member 26 through which the pin can be disposed. In this embodiment, the pin can be disposed through the desired aperture corresponding to the desired minimum length of the plate 10. The pin-and-aperture configuration for the stop 30 is merely one example of a configuration that would allow for variable minimum length selection of the plate 10. It should be understood that the stop 30 can comprise any suitable configuration without departing from the spirit of the present invention. The plate 10, stop 30 and screws 32 preferably comprise a substantially rigid, surgical safe material such as stainless steel, or the like.

FIG. 1 illustrates but one embodiment of the present invention. Other exemplary embodiments include, but are not limited to, a first support member 24 and second support member 26 having a circular or semi-circular cross-section wherein the first support member 24 and the second support member 26 engage at least each other in a manner to allow for length adjustment. Furthermore, more than a first support member 24 and a second support member 26 can be included and can be formed of any suitable size and shape. The embodiments can also include various configurations and locations for points of contact of the anterior cervical corpectomy plate 10 to vertebral bodies comprising the cervical spine 100.

FIG. 2 illustrates the embodiment of the plate 10 of FIG. 1, fixed on a portion of a cervical spine 100. The post-corpectomy cervical spine 100, as illustrated, comprises an intervertebral disc 102 disposed on a vertebral body 104. A graft 106 is disposed between the vertebral body 104 and another vertebral body 108 having an untouched intervertebral disc 110 disposed therebelow. The cervical spine 100 can continue both above and below the illustrated portion with alternating vertebral bodies and vertebral discs. It should be noted that the space between vertebral body 104 and vertebral body 108 can represent the removal of any amount of vertebral bodies and intervertebral disc(s).

The plate 10 is configured to the desired length by fitting the second support member 26 into the channel 22 of the first support member 18 and sliding the second support member 26 axially therein to reach the desired length. The optional stop 30 is disposed in a position to limit compression of the plate 10 to a desired minimum length. The plate 10 is fixed to the cervical spine 100 at the vertebral bodies 104 and 106 above and below the graft 106. More specifically, the first mount 16 is fixed to vertebral body 104 at the apertures 20 by the screws 32. Similarly, the second mount 24 is fixed to the vertebral body 108 at the apertures 28 by the screws 32. As stated above, it is preferable that a plurality of screws 32 are disposed through a plurality of apertures 20 disposed in the first mount 16 and a plurality of apertures 28 disposed in the second mount 24 to fix the plate 10 to the cervical spine 100. The first support member 18 and the second support member 26 axially slide with respect to each other toward each other as the graft 106 settles during fusion. The stop 30 disposed at a pre-determined position along the second support member 26 controls the amount of settling allowed.

It should be emphasized that the above-described embodiments of the present invention, particularly, a “preferred” embodiment, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein with the scope of this disclosure and the present invention and protected by the following claims.

Therefore, having thus described the invention, at least the following is claimed:

1. An anterior cervical corpectomy plate, comprising:
fixing means for fixing the plate to a portion of a cervical spine; and
adjusting means for adjusting a length of the plate.

2. An anterior cervical corpectomy plate, comprising:
a fixing member for fixing the plate to a portion of a cervical spine; and
an adjustable member for adjusting a length of the plate.

3. An anterior cervical corpectomy plate, comprising:
a first plate member having a first mount and a first support member extending from said first mount;
a second plate member having a second mount and a second support member extending from said second mount;
wherein said first mount and said second mount are adapted to each engage a vertebral body and said first support member and said second support member are adapted to engage each other in an axially adjustable manner.

4. An anterior cervical corpectomy plate, comprising:
a first plate member having a first mount and a first support member extending from said first mount;
a second plate member having a second mount and a second support member extending from said second mount;
a plurality of apertures disposed in said first mount and said second mount;
a plurality of screws adapted to engage said plurality of apertures, said screws being adapted to fix said first mount and said second mount each to a vertebral body;
a stop disposed on at least one of said first plate member and said second plate member;
wherein said first support member and said second support member are adapted to engage each other in an axially adjustable manner and said stop is adapted to limit such axial adjustment.
